(19) United States
(12) Patent Application Publication
(10) Pub. No.: US 2023/0250247 A1
(43) Pub. Date: Aug. 10, 2023

(54) SURFACE TREATMENT OF POLY(ARYL ETHER KETONE)S

(71) Applicants: FRAUNHOFER-GESELLSCHAFT ZUR FÖRDERUNG DER ANGEWANDTEN FORSCHUNG e. V., Munchen (DE); SpinPlant GmbH, Halle (Saale) (DE); Hochschule Anhalt, Köthen (Anhalt) (DE)

(72) Inventors: Christian Schmelzer, Halle (Saale) (DE); Andrea Friedmann, Halle (Saale) (DE); Julius Rubers, Halle (Saale) (DE); Tobias Kürbitz, Köthen (Anhalt) (DE); Tobias Hedtke, Halle (Saale) (DE); Christian Herbst, Halle (Saale) (DE)

(21) Appl. No.: 18/105,990

(22) Filed: Feb. 6, 2023

(30) Foreign Application Priority Data
Feb. 8, 2022 (DE) 10 2022102870.4

(51) Int. Cl. C08J 7/12; C08G 65/48; A61L 27/18; A61L 27/22; A61L 27/24; A61L 27/44; C08J 3/24; C08J 7/04

(52) U.S. Cl. CPC C08J 7/12; C08G 65/48; A61L 27/18; A61L 27/222; A61L 27/24; A61L 27/443; C08J 3/24; C08J 7/04; C08J 2371/10; C08J 2489/04; C08K 7/06

(57) ABSTRACT

The present invention relates to a process for the surface treatment of poly(aryl ether ketone)s (PAEKs) comprising the following steps:
Providing an article comprising one or more poly(aryl ether ketone)s (PAEKs);
contacting at least one portion of the surface of the article containing one or more poly(aryl ether ketone)s (PAEKs) with an aldehyde,
   wherein the aldehyde reacts with the poly(aryl ether ketone)(s) (PAEKs) on the at least one portion of the surface of the article to form a hydroxyalkyl and/or hydroxyaryl group,
   a process for functionalizing surface-treated poly(aryl ether ketone)s (PAEKs) comprising the steps of:
a) Treating at least one portion of the surface of an article containing one or more poly(aryl ether ketone)s (PAEKs) with the surface treatment process for poly(aryl ether ketone)s (PAEKs) described herein;
b) Coating the treated at least one portion of the surface of the article with a composition comprising a chemical compound having chemical groups capable of forming a covalent bond with hydroxyalkyl and/or hydroxyaryl groups formed on the surface of the article,
   an article comprising one or more poly(aryl ether ketone)s (PAEKs) and a coating on at least one surface of the article, wherein on the coated at least one portion of the surface of the article the poly(aryl ether ketone)(s) (PAEKs) contains hydroxyalkyl and/or hydroxyaryl groups; and at least one portion of the hydroxyalkyl and/or hydroxyaryl groups of the poly(aryl ether ketone)(s) (PAEKs) has formed covalent bonds with chemical groups of at least one chemical compound in the coating, and
   the use of the article of the invention as described herein as a medical device and/or biotechnological applications, preferably as an implant, scaffold structure for in vitro applications and/or scaffold structure for cell culture applications.

### CROSS REFERENCE TO RELATED APPLICATION(S)

The present application claims priority to German (DE) Patent Application No. 10 2022 102 870.4 filed on Feb. 8, 2022. The entire contents of which are incorporated in its entirety.

### FIELD OF INVENTION

The present invention relates to a method of surface-treating poly(aryl ether ketone)s with aldehydes, a method of functionalizing surface-treated poly(aryl ether ketone)s, an article comprising a functionalized poly(aryl ether ketone), and the use of the article as a medical device and or as a scaffold structure for cell culture applications.

### TECHNICAL BACKGROUND

Poly(ether ether ketone) (PEEK) is a high-performance thermoplastic polymer used for implants in spine, facial and trauma surgery due to its mechanical properties. The material has been approved for medical use for about 20 years, but its widespread use has been delayed.

The reason for this is the surface properties of PEEK, which make it difficult or even impossible to integrate the implant into the surrounding tissue.

Due to the combination of excellent thermal stability, mechanical properties as well as easy processing, the polyaromatic, semi-crystalline, thermoplastic polymer PEEK is used as a material for high-performance applications. It consists of a molecular chain of an aromatic backbone linked by functional ketone and ether groups. The chemical structure of the polyaromatic ketones enables stability at high temperatures above 300° C., resistance to chemical and radiation damage, and compatibility with many reinforcing materials such as glass and carbon fibers.

The main advantage of medical PEEK as an implant material compared to implants made of standard metal alloys is the reduced modulus of elasticity (3-4 GPa), which is close to that of human cortical bone. In addition, PEEK is X-ray and MRI compatible.

After implantation, the water is adsorbed on the implant surface within nanoseconds. The orientation of the polar water molecules on the PEEK surface is influenced by the surface properties of the polymer. As the implant comes into contact with blood plasma (which contains more than 5000 proteins), the subsequent interactions of the proteins with the polymer surface are consequently influenced by the orientation of these initially adsorbed water molecules. The interactions of these proteins with the polymer surface are influenced by the surface chemistry, surface charge, and surface structure, which determine the success or failure of an implant in a particular application. In general, PEEK is characterized by bioinert surface chemistry with hydrophobic properties. These surface properties significantly hinder cell attachment, leading to poor implant integration and consequent implant failure. The biological reaction on the PEEK surface can cause a foreign body reaction, which in turn can lead to loosening of the implant during encapsulation of the implant.

To improve the osseointegration of PEEK implants, biological interactions with components of the extracellular matrix should be enabled. According to the current state of the art, this has been attempted in various approaches by modifying the material surface. For example, surface functionalization by plasma treatments (e.g. with O₂, N₂) as well as coating processes based on a metallic coating (e.g. Ti, TiO₂, Ta), ceramic coating (e.g. zeolite, hydroxyapatite) or biological coating (BMP, morphogenic bone proteins) are used.

However, plasma-based surface functionalization of PEEK leads to only minimal and short-term stable effects with respect to biological interactions.

Due to the good osseointegration of roughened titanium surfaces, a common method to improve the osseointegration of PEEK is to coat PEEK with roughened titanium. Although metallic coatings of PEEK implant surfaces can contribute to better osseointegration, they also bring disadvantages. On the one hand, such a coating causes a significant change in terms of processing, approval and application of the implant as a composite material; on the other hand, the advantage of PEEK in terms of radiological diagnostics is compromised.

Furthermore, the premature wear of such coatings has been described.

In order to be able to realize biological coatings (silk fibroin, BMP) on bioinert PEEK surfaces, these are etched e.g., with sulfuric acid, which roughens the PEEK surface.

Functionalization of PEEK surfaces with respect to biological interactions, for example to significantly improve binding to protein without affecting the surface structure of the PEEK, is not known in the prior art—as shown in the three examples listed.

The present invention describes a method of pretreatment of poly(aryl ether ketone)s (PAEKs), preferably PEEK, with aldehydes. The pretreatment of PAEK surfaces described herein enables functionalization through covalent chemical bonding. In particular, this method can be used for pretreatment prior to coating PAEKs with biomolecules but is not limited thereto.

### SUMMARY OF THE INVENTION

The present invention relates to a process for the surface treatment of poly(aryl ether ketone)s (PAEKs) comprising the following steps:
Providing an article comprising one or more poly(aryl ether ketone)s (PAEKs);
contacting at least one portion of the surface of the article containing one or more poly(aryl ether ketone)s (PAEKs) with an aldehyde,

wherein the aldehyde reacts with the poly(aryl ether ketone)(s) (PAEKs) on the at least one portion of the surface of the article to form a hydroxyalkyl and/or hydroxyaryl group.

Further, the present invention relates to a process for functionalizing surface-treated poly(aryl ether ketone)s (PAEKs) comprising the following steps:
a) Treating at least one portion of the surface of an article containing one or more poly(aryl ether ketone)s (PAEKs) with the surface treatment process for poly(aryl ether ketone)s (PAEKs) described herein;
b) Coating the treated at least one portion of the surface of the article with a composition comprising a chemical compound having chemical groups capable of forming a covalent bond with the hydroxyalkyl and/or hydroxyaryl groups formed on the surface of the article.

Further, the present invention relates to an article comprising one or more poly(aryl ether ketone)s (PAEKs) and a coating on at least one portion of the surface of the article, wherein on the coated at least one portion of the surface of the article, the poly(aryl ether ketone)(s) (PAEKs) contains hydroxyalkyl and/or hydroxyaryl groups; and

at least one portion of the hydroxyalkyl and/or hydroxyaryl groups of the poly(aryl ether ketone)(s) (PAEKs) have formed covalent bonds with chemical groups of at least one chemical compound in the coating.

Finally, the present invention relates to the article of the invention as described herein being a medical device and/or biotechnological applications, preferably an implant, scaffold structure for in vitro applications and/or scaffold structure for cell culture applications.

### DETAILED DESCRIPTION OF THE INVENTION

### Process for the Surface Treatment of Poly(Aryl Ether Ketone)s (PAEK)

In a first aspect, the present invention relates to a method for surface treatment of poly(aryl ether ketone)s (PAEK) comprising the following steps:
Providing an article comprising one or more poly(aryl ether ketone)s (PAEKs);
contacting at least one portion of the surface of the article containing one or more poly(aryl ether ketone)s (PAEKs) with an aldehyde,

wherein the aldehyde reacts with the poly(aryl ether ketone)(s) (PAEKs) on the at least one portion of the surface of the article to form a hydroxyalkyl and/or hydroxyaryl group.

The polymer backbone of poly(aryl ether ketone)s (PAEKs) consists of 1,4-substituted aryl groups (R) linked by ketone (R—CO—R) and/or ether groups (R—O—R).

The poly(aryl ether ketone)s (PAEKs) are preferably selected from poly(ether ketone) (PEK), poly(ether ether ketone) (PEEK), poly(ether ketone ketone) (PEKK), poly(ether ether ether ketone) (PEEEK), poly(ether ether ketone ketones) (PEEKK), poly(ether ketone ether ketone ketones) (PEKEKK) or mixtures thereof. Poly(ether ether ketone) (PEEK) is particularly preferred.

Poly(ether ether ketone) (PEEK) has the general chemical formula (—R—O—R—O—R—CO—)ₙ.

Poly(ether ketone) (PEK) has the general chemical formula (—O—R—CO—R—)ₙ.

Poly(ether ketone ketone) (PEKK) has the general chemical formula (—R—O—R—CO—CO—)ₙ.

Poly(ether ether ether ketone) (PEEEK) has the general chemical formula (—R—O—R—O—R—O—CO—)ₙ.

Poly(ether ether ketone ketone) (PEEKK) has the general chemical formula (—R—O—R—O—R—CO—R—CO—)ₙ.

Poly(ether ketone ether ketone ketone) (PEKEKK) has the general chemical formula (—R—O—R—CO—R—O—R—CO—R—CO—)ₙ.

In each case, R represents a 1,4-substituted aryl group and n represents the number of repeats of the respective monomer.

The article may comprise one or more poly(aryl ether ketone)s (PAEKs), such as one poly(aryl ether ketone) (PAEK) or a mixture of two to five, such as two or three poly(aryl ether ketone)s (PAEKs). Preferably, the article comprises one poly(aryl ether ketone) (PAEK).

The article may consist of one or more poly(aryl ether ketone)s (PAEKs), such as one poly(aryl ether ketone) (PAEK) or a mixture of two to five, such as two or three poly(aryl ether ketone)s (PAEKs). Preferably, the article consists of one poly(aryl ether ketone) (PAEK).

In another embodiment, the article comprises a blend of one or more poly(aryl ether ketone)s (PAEKs) with an inorganic structural material, preferably glass fibers, carbon fibers, and/or hydroxyapatite. In this regard, the article can comprise one or more poly(aryl ether ketone) (PAEKs), such as one poly(aryl ether ketone) (PAEK) or a mixture of two to five, such as two or three poly(aryl ether ketone)s (PAEKs). Preferably, the article comprises one poly(aryl ether ketone) (PAEK).

The weight percentage of inorganic structural material is preferably in the range from 10 to 50 wt %, more preferably in the range from 20 to 30 wt %, based on the total weight of the article. The weight percentage of the poly(aryl ether ketone)(s) (PAEKs) is preferably in the range of 50 to 90 wt %, more preferably in the range of 70 to 80 wt %, based on the total weight of the article.

The article may have any shape. Preferably, the article is already formed into the shape in which it is to be used after treatment with the process(es) according to the invention.

It is important that regardless of the weight percentage of poly(aryl ether ketone)(s) (PAEK), the surface(s) of the article to be treated have poly(aryl ether ketone) groups that can react with aldehyde.

At least one portion of the surface, preferably the entire surface of the article is contacted with an aldehyde.

The aldehyde is preferably a linear or branched aliphatic or aromatic hydrocarbon, preferably a linear aliphatic hydrocarbon, having 1 to 12, more preferably 1 to 7, even more preferably 1 to 5 carbon atoms with 1 to 4, preferably 1 or 2 aldehyde groups.

The aldehyde is preferably selected from formaldehyde (methanol), glyoxal (ethanedial), succinaldehyde (butanedial) and glutaraldehyde (pentanedial) or mixtures thereof.

Formaldehyde is particularly preferred.

The aldehyde is preferably present as a solution, preferably as an aqueous solution. This solution typically contains the aldehyde in a concentration of 25 to 75%, preferably 30 to 55%.

The aldehyde may be contacted with the at least one portion of the surface of the article as a liquid, as a gas, or as an aerosol.

Typically, this step occurs over a limited period of time, such as 10 min to 60 min, preferably 15 min to 45 min, more preferably 30 min.

In this regard, the article may be immersed in a liquid aldehyde solution. The temperature of the liquid plays a minor role and is preferably in the range of 10 to 50° C., preferably 15 to 30° C. (usually depending on the boiling point of the solution).

After the contact time has elapsed, the article is preferably removed from the liquid aldehyde solution and the excess aldehyde solution is removed from the at least one surface, for example by wiping or blotting.

Upon contacting the at least one portion of the surface of the article with the aldehyde in gaseous or aerosol form, the article is preferably placed in a sealed fumigation room, such as a fumigation chamber or box, into which the aldehyde gas or aerosol is injected.

At the end of the contact time, the aldehyde gas or aerosol is evaporated from the fumigation room and the article is removed from the fumigation room. Any excess aldehyde is preferably removed from the at least one portion of the surface, for example by wiping or blotting.

The contact of the poly(aryl ether ketone) groups on the at least one portion of the surface of the article with the aldehyde results in a chemical reaction of the aldehyde with the poly(aryl ether ketone) groups, preferably with hydrocarbon groups of the poly(aryl ether ketone) groups, in particular with hydrocarbon groups of the aryl groups of the poly(aryl ether ketone) groups on the at least one portion of the surface of the article. Thereby, a hydroxyalkyl and/or hydroxyaryl group is preferably formed from the hydrocarbon group and the aldehyde group, preferably a methylolyl group is added to the hydrocarbon group.

These so formed hydroxyalkyl and/or hydroxyaryl groups, preferably the so added methyloyl groups, on the at least one portion of the surface of the article are suitable for forming a covalent bond with suitable chemical groups of a coating in a further process of the present invention.

The at least one portion of the surface of the article comprising one or more poly(aryl ether ketone)s (PAEKs) may additionally be subjected to at least one further surface treatment, such as a plasma treatment with, for example O₂ or N₂. Such a plasma treatment may increase the number of reactive binding sites on the at least one portion of the surface of the article for covalent bonds with suitable chemical groups of a coating.

The plasma treatment may be performed in a process step subsequent to the process step of contacting at least one portion of the surface of the article comprising poly(aryl ether ketone) (PAEK) with an aldehyde.

The plasma treatment may be performed in a process step prior to the process step of contacting at least one portion of the surface of the article comprising poly(aryl ether ketone) (PAEK) with an aldehyde. This may increase the adhesion of the coating to the at least one portion of the surface of the article.

### Process for the Functionalization of Surface-Treated Poly(Aryl Ether Ketone)s (PAEKs)

In another aspect, the present invention relates to a process for functionalizing surface-treated poly(aryl ether ketone)s (PAEKs) comprising the following steps:
a) Treating at least one portion of the surface of an article comprising one or more poly(aryl ether ketone) (PAEKs) with the surface treatment process for poly(aryl ether ketone)s (PAEKs) described herein;
b) Coating the treated at least one portion of the surface of the article with a composition comprising a chemical compound having chemical groups capable of forming a covalent bond with hydroxyalkyl and/or hydroxyaryl groups formed on the surface of the article.

Preferably, any aspect described herein and any embodiment described herein of the method of surface treatment of poly(aryl ether ketone)s (PAEKs) according to the invention is applicable to the surface treatment of the at least one portion of the surface of the article comprising one or more poly(aryl ether ketone)s (PAEKs).

The composition for coating the treated at least one portion of the surface of the article preferably comprises biological material or biomolecules.

Preferred are natural, artificial, chemically modified or biotechnologically produced biological material or biomolecules.

“Natural” in this context means that the biological material or biomolecules are derived from natural sources.

“Artificial” in this context means that the biological material or biomolecules were produced via chemical processes outside of natural sources.

“Chemically modified” means that biological material or biomolecules have been modified by chemical or biotechnological process steps.

“Biotechnologically produced” means that the biological material or biomolecules have been produced via biotechnological processes.

“Biological material” means material of biological (i.e. plant, fungal or animal) origin. It may be a single class of molecules of biological origin or a mixture of several classes of molecules. Plant, fungal or animal cells or tissues also fall within the definition of biological material.

Suitable biological material or biomolecules are preferably selected from proteins, oligo- or polypeptides, amino acids, mono-, oligo- or polysaccharides, proteoglycans, glycoproteins, glycosaminoglycans, lipids, glycolipids, nucleotides, vitamins and other low molecular weight compounds (i.e. compounds having a molecular mass of not more than 800 g/mol) or mixtures thereof.

The composition preferably comprises at least proteins or protein mixtures, preferably proteins or protein mixtures of the animal extracellular matrix, such as gelatin or collagen.

For the process according to the invention, it is necessary that the composition comprises at least one chemical compound having chemical groups capable of forming a covalent bond with the hydroxyalkyl and/or hydroxyaryl groups formed on the surface of the article.

Such chemical groups include amino groups, alcohol groups, aldehyde groups, carboxyl groups, halide groups, and mixtures thereof.

These chemical groups are preferably present in the biological materials or biomolecules described above. The chemical groups may have already been introduced into the biological materials or biomolecules during the natural, chemical or biotechnological synthesis thereof or by chemical modification thereof. Particularly preferably, such chemical groups are found in the amino acid side chains of proteins or protein mixtures. For example, primary amines (e.g., from lysine side chains) and hydroxyphenyl groups (e.g., from tyrosine side chains) may form covalent bonds with the hydroxyalkyl and/or hydroxyaryl groups, preferably the added methylolyl groups, of the surface-treated poly(aryl ether ketone)s (PAEKs) via a methylene group (—CH₂—).

Thus, a covalent bond can be formed between the chemical compounds, preferably the biological material or biomolecules, the coating and the one or more surface-treated poly(aryl ether ketone)s (PAEKs) on the at least one portion of the surface of the article.

The chemical compounds of the coating are preferably tissue compatible (biocompatible). A covalent bond of the coating with the at least one portion of the surface of the article can thus improve the osseointegration of the article when being, for example, an implant.

By coating and covalently bonding the coating to the one or more surface-treated poly(aryl ether ketone)s (PAEKs) of the article of the inventive process, coatings of the poly(aryl ether ketone)s (PAEKs) with metallic or ceramic materials such as Ti, TiO₂, Ta, zeolite or hydroxyapatite can be omitted.

Preferably, all surfaces of the article are first surface treated by the process described above and then coated by the process now described.

The coating can be applied by any conceivable process.

Preferably, the treated at least one portion of the surface of the article is coated with the composition by electrospinning, electrospray, aerosol deposition, doctoring, dip coating, or spray coating.

Particularly preferred is a coating by electrospinning, for example, with nanofibers of the composition, preferably nanofibers of peptides or proteins, such as collagen or gelatin.

In electrospinning experiments, it was found that a more stable coating can be obtained on the one or more surface-treated poly(aryl ether ketone)s (PAEKs) of the article when the article was not in direct contact with the collector electrode of the electrospinning apparatus.

The coating thickness is typically in the range of 0.1 to 500 μm, preferably from 0.5 to 400 μm, more preferably from 1 to 300 μm.

In electrospinning nanofibers, the nanofibers typically have an average diameter in the range of 10 to 700 nm, preferably 25 to 500 nm, more preferably 50 to 300 nm.

Preferably, the coating is applied in close temporal relation to the surface treatment of the at least one portion of the surface of the article according to the surface treatment process described above. This can prevent the hydroxyalkyl and/or hydroxyaryl groups formed in the surface treatment process, preferably the added methyloyl groups, from entering into unwanted chemical reactions, for example with reactive groups in the vicinity or with themselves, and thus being unavailable for covalent bonding with the chemical groups of the coating.

After forming the covalent bond to the one or more surface-treated poly(aryl ether ketone)s (PAEKs), the composition of the coating may be further cross-linked. For this purpose, remaining reactive chemical groups of the composition that have not formed a covalent bond with the one or more surface-treated poly(aryl ether ketone)s (PAEKs) may be contacted with reactive compounds, such as aldehydes, preferably formaldehyde, resulting in cross-linking of the chemical groups. Further suitable cross-linking can be achieved via cross-linking agents from the group of carbodiimides, such as 1-ethyl-3-(3-dimethylaminopropyl)carbodiimide (EDC), in combination with oxidizing agents, such as N-hydroxysuccinimide (NHS) or N-hydroxysulfosuccinimide sodium salt (sulfo-NHS).

### Article and Use

In another aspect, the present invention relates to an article comprising one or more poly(aryl ether ketone)s (PAEKs) and a coating on at least one portion of the surface of the article, wherein on the coated at least one portion of the surface of the article, the poly(aryl ether ketone)(s) (PAEKs) contain(s) hydroxyalkyl and/or hydroxyaryl groups; and at least one portion of the hydroxyalkyl and/or hydroxyaryl groups of the poly(aryl ether ketone)(s) (PAEKs) have formed covalent bonds with chemical groups of at least one chemical compound in the coating.

The article is preferably preparable by the process of functionalizing surface-treated poly(aryl ether ketone)s (PAEKs).

Preferably, the hydroxyalkyl and/or hydroxyaryl groups are formed on the at least one portion of the surface of the article by the process for the surface treatment of poly(aryl ether ketone)s (PAEKs).

Preferably, any aspect and embodiment described herein of the inventive process for the surface treatment of poly(aryl ether ketone)s (PAEKs) and/or the inventive process for functionalizing surface-treated poly(aryl ether ketone)s (PAEKs) is applicable.

The coating is covalently bonded to the at least one portion of the surface of the article. This results in sufficient structural integrity between the at least one portion of the surface of the article and the coating to prevent detachment of the coating, caused for example by shrinkage.

The article is preferably cytocompatible. This property is preferably produced by the coating. It is shown that due to the increased cytocompatibility, an improved adhesion of cells to the poly(aryl ether ketone) material can be observed. This improves the osseointegration of PAEK implants.

The article is preferably used as a medical device and/or biotechnological application, preferably as an implant, a scaffold structure for in vitro applications and/or a scaffold structure for cell culture applications.

The object is particularly suitable as an orthopaedic or spinal implant.

### Advantages of the Invention

The processes of the invention can be used to covalently bond coating materials to the surface of poly(aryl ether ketone) (PAEKs)-containing molded articles that have different chemical and/or physical properties compared to poly(aryl ether ketone)s (PAEKs). For example, as described herein, cytocompatible coatings can be covalently established on the surface of poly(aryl ether ketone)s (PAEKs)-containing molded articles using the methods of the invention described herein.

In addition, the processes according to the invention allow the coating of poly(aryl ether ketone) (PAEKs)-containing molded articles while maintaining the structural properties of the poly(aryl ether ketones) (PAEKs).

By coating with a cytocompatible coating, such as a protein- or biopolymer-containing coating, improved adhesion of cells to the poly(aryl ether ketone) material can be observed.

This improves the osseointegration of PAEK implants.

The poly(aryl ether ketone) (PAEK)-containing molded articles according to the invention are thus particularly suitable for medical applications and scaffold structures in cell culture and tissue engineering applications. The poly(aryl ether ketone) (PAEK)-containing molded articles according to the invention are particularly suitable as implants, in particular as orthopaedic or spinal implants.

### DESCRIPTION OF THE FIGURE

FIG. 1 shows a scanning electron microscope image of the boundary phase of a formaldehyde surface-treated molded article of PEEK (FA-etched PEEK) and a gelatin coating formed by electrospinning gelatin nanofibers (intermediate nanofiber layer) on the surface of the surface-treated molded article. The arrows in the FIGURE show the contact area where the fibers of the coating fuse with the formaldehyde treated surface of the molded article by covalent bonding. This fusion provides sufficient structural integrity between the surface of the PEEK molded article and the fibre coating to prevent detachment of the coating, for example by shrinkage.

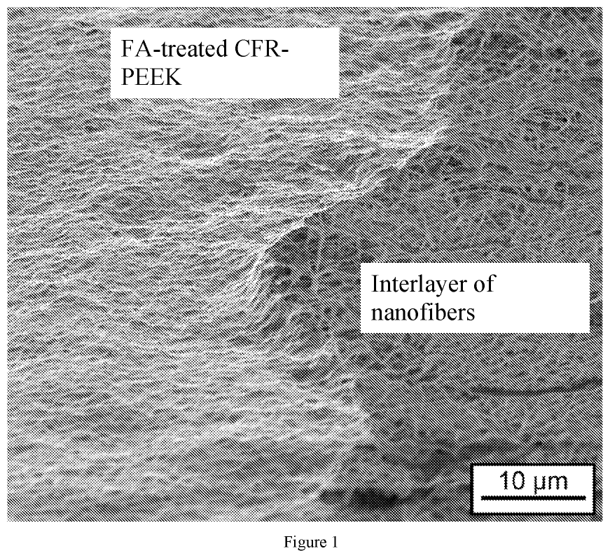

### EXAMPLES

### Example 1: Application of a Stable Coating of Non-Oriented Gelatine Nanofibers on CFR-PEEK Samples

Carbon fiber-reinforced poly(ether ether ketone) (CFR-PEEK) with 30% carbon fibers from POLYTRON Kunststofftechnik (Victrex® PEEK 150CA30, Bergisch Gladbach, Germany) was used for the coating experiments. Test articles with a surface area of 484 mm² and a thickness of 1 mm were manufactured from the material. The CFR-PEEK test articles were cleaned with ddH₂O and then immersed in a 37% formaldehyde solution (Carl Roth, Germany) at room temperature. After 30 minutes, the articles were removed from the formaldehyde solution and the excess liquid was removed with a paper towel. The molded articles were coated with protein nanofibers immediately after the formaldehyde activation. For nanofiber coating, the formaldehyde-activated CFR-PEEK molded articles were placed on a plate collector in a custom-built electrospinning experimental setup. 20% (w/v) gelatin was dissolved in 50% (v/v) acetic acid and transferred into a 20 mL syringe (B. Braun Perfusor). The outlet of the syringe was connected to a 21 G blunt cannula via infusion tubing (B. Braun Perfusor). The syringe was placed in a syringe pump (neMESYS, Cetoni GmbH, Germany) with a software controlled feed rate. The syringe was connected to a high voltage DC power source (Heinzinger, Germany) and placed over a grounded copper plate with an area of 10×10 cm² at a vertical distance of 12 cm. The voltage was set at 12 kV and the injection rate was set to 5 μL/min. On each formaldehyde activated CFR-PEEK molded article, 0.25 mL of gelatin solution was deposited by electrospinning.

The CFR-PEEK test articles coated with nanofibers were then dried at 37° C. for 24 hours. The test articles were then placed in a desiccator (total volume approx. 2.4 L) over a reservoir of 37% formaldehyde solution in water. For every 30 mg of gelatin (dry weight), 10 mL of formaldehyde solution was used. The samples were incubated in the desiccator for 105 min to stabilize the gelatin fiber coating applied via electrospinning.

This process results in an intermediate layer of fibres that adhere firmly to the CFR-PEEK surface. To remove this layer completely, it must be scraped or abraded. The fibers deposited on this interlayer are effectively cross-linked and form a stable coating with an average thickness of 211±49 μm and a dry mass of 3.1±0.3 mg. The average diameter of the deposited fibers was determined by scanning electron microscopy and image analysis to be 143±29 nm for untreated fibers and a slight increase to 155±34 nm after formaldehyde crosslinking.

This process leads to a less efficient coating of PEEK (without additional material such as glass fibre, carbon fibres or the like), as the homogeneity of the electric field was compromised by direct contact with the collector electrode, which increased the insulating properties of PEEK. The efficiency of the PEEK coating can be significantly improved by modifying the electrospinning system as described in Example 5.

### Example 2: Application of a Stable Coating of Oriented Gelatine Nanofibres on CFR-PEEK Test Specimens

CFR-PEEK test articles were prepared and pre-activated as described in Example 1. After formaldehyde pretreatment, the test articles were placed in an electrospinning device between two grounded copper collector plates, each having an area of 2×1 cm². Subsequently, the test articles were coated with gelatin nanofibers by electrospinning as described in Example 1. The modified collector setup resulted in a coating with oriented fibers.

### Example 3: Improved Cytocompatibility and Cell Proliferation

CFR-PEEK test articles were cut to 10×10×1 mm³ dimensions and then sanded with 500 grit sandpaper to achieve a surface roughness of 0.37 μm. The test articles were pre-activated by incubation in 37% formaldehyde solution as described in Example 1. The samples were then coated with gelatin nanofibers by electrospinning as described in Example 1. For this procedure, 0.1 mL of gelatin solution was electrospun and applied to six test articles. After electrospinning, the test articles were dried at 37° C. for 24 hours before the fibers were cross-linked by formaldehyde fumigation as described in Example 1. To optimize the test articles for cell culture experiments, the test articles were stored at 50° C. and 80 mbar for 48 h and placed in a 24-hole plate. Each article was disinfected by immersing it in 1 mL of 70% ethanol for 2 h at room temperature under a biological workbench. The ethanol was rinsed from the articles with sterile phosphate buffered saline (PBS). The articles were stored in 1 mL of Delbecco's Modified Eagle Medium F-12 (DMEM/F-12) at 37° C. for 24 h before cell seeding.

Human chondrosarcoma cells (SW1353) were cultured to confluence in a culture medium consisting of DMEM/F-12, 10% fetal bovine serum (FBS), 1% L-glutamine, and 1% penicillin/streptomycin at 37° C. in a humid atmosphere at 5% CO₂. The Cells were seeded in polystyrene perforated plates (control), on untreated and on gelatin-coated CFR-PEEK samples (n=6) at a density of 5000 cells/cm². Cell viability assays (CellTiter-Blue, Promega) were performed on days 0, 1, 2, 3, 4, 7 and 8, respectively. Cell viability in the control groups was ≥97.8%. The cell count on the gelatin-coated CFR-PEEK test articles was approximately 33% higher after 192 hours compared to the cell count on the untreated CFR-PEEK test articles. Compared to Example 1, the modified coating procedure described herein resulted in a large increase in cytocompatibility.

### Example 4: Alternative Cross-Linking Strategy

CFR-PEEK test articles (22×22×1 mm³) were prepared as described in Example 1, cleaned, and immersed in 37% formaldehyde solution for 30 minutes at room temperature. The test articles were then removed from the liquid. The test articles were then dried and coated with gelatin fibers by electrospinning as described in Example 1. After electrospinning, the fiber coated CFR-PEEK samples were dried at 37° C. for 24 hours. The fibers were stabilized by chemical cross-linking with 1-ethyl-3-(3-dimethylaminopropyl)carbodiimide hydrochloride (EDC-HCl, Merck, Germany). 100 mM EDC-HCl was dissolved in isopropanol. 3 mL of EDC solution per mg of gelatin fibers (dry weight) was added to a small container. The CFR-PEEK test articles coated with fibers were then immersed in the EDC solution in the container for 2 hours at room temperature.

The average fiber diameter of the deposited and EDC-crosslinked fibers was 450±34 nm (determined by scanning electron microscopy and image analysis). An interlayer formed, which strongly adhered to the CFR-PEEK surface and had to be scraped or abraded for complete removal. However, the cross-linking of the fibers deposited on this interlayer was less effective than in Example 1.

### Example 5: Application of Devices on a Production Scale

PEEK and CFR-PEEK test articles were prepared as described in Example 1 and inserted into a Nanospider NS 1WS 500 U electrospinning device (Elmarco, Czech Republic). For this purpose, the test articles were fixed to the polypropylene substrate with double-sided adhesive tape. A protein/polymer solution containing native collagen, poly(ethylene oxide) and hydroxyapatite (SpinPlant GmbH, Germany) was electrospun at 80 kV for 30 minutes and applied to the CFR-PEEK test articles. Since the articles were not in direct contact with the collector electrode, the nanofibers were deposited equally efficiently on PEEK and CFR-PEEK specimens.

### Example 6: 3D Coating of CFR-PEEK Implants

A block of CFR-PEEK was fabricated and pre-activated by immersion in a 37% formaldehyde solution as described in Example 1. The block was then mounted on a custom-made spinning device and this was placed in the electrospinning apparatus described in Example 1 between the grounded copper collector plate and the needle connected to the high voltage DC power source. The electrospinning was performed as described in Example 1. In this case, the spinning device with the CFR-PEEK block was rotated at a speed of about 10 rpm. This resulted in a homogeneous coating of all sides of the CFR-PEEK block. Subsequent cross-linking by formaldehyde fumigation as described in Example 1 resulted in a stable coating.

What is claimed is:

1. A process for the surface treatment of poly(aryl ether ketone)s (PAEKs) comprising the following steps:
Providing an article comprising one or more poly(aryl ether ketone)s (PAEKs);
contacting at least one portion of the surface of the article comprising poly(aryl ether ketone) (PAEK) with an aldehyde,
wherein the aldehyde reacts with the poly(aryl ether ketone)(s) (PAEKs) on the at least one portion of the surface of the article to form a hydroxyalkyl and/or hydroxyaryl group.

2. The process of claim 1, wherein the poly(aryl ether ketone)s (PAEKs) are selected from poly(ether ketone) (PEK), poly(ether ether ketone) (PEEK), poly(ether ketone ketone) (PEKK), poly(ether ether ether ketone) (PEEEK), poly(ether ether ketone ketone) (PEEKK), poly(ether ketone ether ketone ketone) (PEKEKK), or mixtures thereof.

3. The process according to claim 1, wherein the article comprising one or more poly(aryl ether ketone)s (PAEKs) comprises a blend of the poly(aryl ether ketone)(s) (PAEKs) with an inorganic structural material.

4. The process according to claim 3, wherein the inorganic structural material is selected from glass fibers, carbon fibers, hydroxyapatite, or mixtures thereof.

5. The process according to claim 1, wherein the aldehyde is selected from formaldehyde (methanol), glyoxal (ethanedial), succinaldehyde (butanedial) and glutaraldehyde (pentanedial) or mixtures thereof.

6. The process according to claim 1, wherein the aldehyde is contacted as a liquid, a gas or an aerosol with at least one portion of the surface of the article comprising one or more poly(aryl ether ketone)s (PAEKs).

7. The process according to claim 1, wherein the at least one portion of the surface of the article comprising one or more poly(aryl ether ketone)s (PAEKs) is additionally subjected to at least one further surface treatment, such as a plasma treatment.

8. A process for functionalizing surface-treated poly(aryl ether ketone)s (PAEKs) comprising the steps of:
a) Treating at least one portion of the surface of the article comprising one or more poly(aryl ether ketone)s (PAEKs) with the process according to claim 1;
b) Coating the treated at least one portion of the surface of the article with a composition comprising a chemical compound having chemical groups capable of forming a covalent bond with the hydroxyalkyl and/or hydroxyaryl groups formed on the surface of the article.

9. The process according to claim 8, wherein the composition comprises biological material or biomolecules.

10. The process according to claim 9, wherein the biological material or biomolecules is/are selected from natural, artificial, chemically modified or biotechnologically produced biological material or biomolecules.

11. The process according to claim 9, wherein the biological material or biomolecules is/are selected from proteins, oligo- or polypeptides, amino acids, mono-, oligo- or polysaccharides, proteoglycans, glycoproteins or glycosaminoglycans, lipids, glycolipids, nucleotides, vitamins and other low molecular weight compounds or mixtures thereof.

12. The process according to claim 9, wherein the biological material or biomolecules is/are selected from collagen or gelatin.

13. The process according to claim 8, wherein the chemical groups capable of forming a covalent bond with the hydroxyalkyl and/or hydroxyaryl groups formed on the surface of the article are selected from amino groups, alcohol groups, aldehyde groups, carboxyl groups, halide groups, or mixtures thereof.

14. The process according to claim 8, wherein the treated at least one portion of the surface is coated with the composition by electrospinning, electrospray, aerosol deposition, doctoring, dip coating or spray coating.

15. The process according to claim 8, wherein after coating the composition is cross-linked.

16. An article comprising one or more poly(aryl ether ketone)s (PAEKs) and a coating on at least one portion of the surface of the article, wherein
on the coated at least one portion of the surface of the article, the poly(aryl ether ketone)(s) (PAEKs) contain(s) hydroxyalkyl and/or hydroxyaryl groups; and
at least one portion of the hydroxyalkyl and/or hydroxyaryl groups of the poly(aryl ether ketone)(s) (PAEKs) have formed covalent bonds with chemical groups of at least one chemical compound in the coating.

17. The article of claim 16, made by the process of claim 8.

18. The article of claim 16, wherein the article is cytocompatible.

19. The article according to claim 16 being a medical device and/or biotechnological application.

20. The article according to claim 19 being an implant, scaffold structure for in vitro applications and/or scaffold structure for cell culture applications
